(19) United States
(12) Patent Application Publication
(10) Pub. No.: US 2024/0190796 A1
(43) Pub. Date: Jun. 13, 2024

(54) METHOD FOR SEPARATING ALL OR SOME OF THE COMPOUNDS FROM A BIOGAS IN THE LIQUID STATE OR IN THE TWO-PHASE STATE

(71) Applicant: Sublime Energie, Paris (FR)

(72) Inventors: Chourouk Nait Saidi, Palaiseau (FR); Rodrigo Rivera Tinoco, PV Venlo (NL)

(21) Appl. No.: 18/554,164

(22) PCT Filed: Sep. 30, 2022
(86) PCT No.: PCT/EP2022/077360

§ 371 Date: Oct. 5, 2023

(30) Foreign Application Priority Data
Oct. 1, 2021 (FR) FR2110431
Nov. 8, 2021 (FR) FR2111835

(51) Int. Cl. C07C 7/08; C01B 32/50; F25J 3/02

(52) U.S. Cl. CPC C07C 7/08; C01B 32/50; F25J 3/0209

(57) ABSTRACT

A method is used for separating all or some of the compounds from a biogas in the liquid or the two-phase liquid/vapor state containing methane, CO₂ and optionally hydrocarbon(s) from the C₃ to C₇ family. The methane is separated from the other compounds by cryogenic distillation by injecting, into a distillation column, the liquefied biogas at an equilibrium temperature that makes it possible to obtain a two-phase mixture, ensuring the separation of the different compounds, and a liquefying agent, in the liquid state, composed of a hydrocarbon or a mixture of hydrocarbon(s) from the C₃ to C₇ family. The liquefying agent is injected at the top of the column, above the biogas inlet, at a temperature lower than or equal to the CO₂ desublimation temperature at a given pressure of the column and in an amount proportional to the vapor flow rate of the CO₂ ascending at the top of the column.

### CROSS REFERENCE TO RELATED APPLICATIONS

This application is a national phase entry under 35 U.S.C. § 371 of International Patent Application PCT/EP2022/077360, filed Sep. 30, 2022, designating the United States of America and published as International Patent Publication WO 2023/052624 A1 on Apr. 6, 2023, which claims the benefit under Article 8 of the Patent Cooperation Treaty to French Patent Application Serial No. FR2110431, filed Oct. 1, 2021, and to French Patent Application Serial No. FR2111835, filed Nov. 8, 2021.

### TECHNICAL FIELD

The present disclosure relates to the field of biogas recovery, and relates more particularly to a method for separating all or some of the compounds from a biogas in the liquid/vapor two-phase state, the biogas containing methane, carbon dioxide and optionally hydrocarbons from the family C₃ to C₇ allowing its liquefaction.

The method according to the present disclosure is intended in particular, but not exclusively, to purify the biogas in the liquid state or in the two-phase liquid/vapor state for the purposes of recovering all or some of its compounds, and in particular methane to be used as fuel for vehicles or to be injected into a natural gas network and carbon dioxide.

### BACKGROUND

Among the methods for purifying the known biogas, the methods conventionally used to separate methane from carbon dioxide are membrane separation, PSA (pressure swing adsorption) and cryogenic distillation.

Membrane separation is based on the use of membranes made of materials allowing carbon dioxide to pass and retaining methane. PSA is based on the adsorption of carbon dioxide into the solid components of the high-pressure adsorption column and a low-pressure desorption. The cryogenic distillation is based on successive lowering of the temperature until liquid carbon dioxide and liquefied biomethane are produced or the carbon dioxide is desublimated, which produces liquid carbon dioxide and bio-methane gas.

These methods as conventionally used have disadvantages for separating a biogas in the liquid state or in the two-phase state. Either they do not make it possible to achieve satisfactory recovered methane purity levels, or they do not make it possible to recover carbon dioxide, or they prove unsuitable for treating a liquefied biogas or one containing hydrocarbons, in particular butane, used to liquefy the biogas. Thus, the membrane separation technique is not to be preferred when the biogas comprises hydrocarbons, and in particular butane, the latter degrading the membranes quickly. PSA does not allow the desired purity levels to be achieved. Furthermore, this method requires the contribution of a large amount of heat, which goes against the use of cold previously spent to liquefy the biogas. The cryogenic distillation technique has the disadvantage of producing solid carbon dioxide particles (dry ice crystals) that can create blockages in the heat exchangers and the packing material in the top of the column.

Methods for separating compounds are also known from patent applications U.S. Pat. No. 4,318,723 and US 2003/0047492. However, the methods described relate to the separation of the compounds of a natural gas, and do not address the problem of separation of the compounds of a biogas and of recovering each of these compounds at purity levels on the order of 99.9%.

The present disclosure aims to remedy these problems by proposing a separation method making it possible to separate the compounds from a biogas in the liquid state or in the two-phase liquid/vapor state so as to recover the methane at a high purity level as well as to recover carbon dioxide by avoiding the problems of clogging and formation of dry ice.

### BRIEF SUMMARY

To this end, and according to a first aspect, the present disclosure proposes a method for separating all or some of the compounds from a biogas in the liquid state or in the two-phase liquid/vapor state, containing methane, carbon dioxide and optionally a hydrocarbon or a mixture of hydrocarbons from the C₃ to C₇ family, the method being remarkable in that a first separation for separating the methane from the other compounds is carried out by cryogenic distillation in a first distillation column comprising a column top brought to the condensation temperature of the methane at a given pressure, the first separation being carried out by injecting into the first distillation column:
in the primary feed, the biogas liquefied at an equilibrium temperature making it possible to obtain a two-phase mixture ensuring the separation of the various compounds, and
in the secondary feed, a liquefying agent in the liquid state composed of a hydrocarbon or a mixture of hydrocarbon(s) from the C₃ to C₇ family, the liquefying agent being injected at the top of the column, above the biogas inlet, at a temperature T1 lower than or equal to the carbon dioxide desublimation temperature at a given pressure of the distillation column and in an amount proportional to the vapor flow rate of the carbon dioxide ascending to the top of the column.

The injection of a liquefying agent at the top of the column above the biogas inlet at a determined temperature and amount, namely at a temperature lower than or equal to the temperature at which the carbon dioxide would desublimate in the distillation column when kept at a given pressure on the one hand, and in an amount proportional to the vapor flow rate of the carbon dioxide ascending to the top of the column, thus makes it possible to dissolve the dry ice crystals that form at the top of the distillation column used to separate the methane from the other compounds by ensuring a satisfactory separation of the methane gas from the liquid mixture composed of carbon dioxide and of the liquefying agent.

The term “desublimation temperature” denotes the temperature at which the desublimation (going from the gas state to the solid state) of the carbon dioxide is caused. The desublimation temperature may also be referred to as the solid condensation, crystallization, or even reverse sublimation temperature.

Advantageously, the liquefying agent is injected at the methane reflux.

Advantageously, the liquefying agent is injected into the first distillation column at a temperature T1 on the order of −100° C.

Advantageously, the liquefying agent is subjected to cooling in two stages under pressure before it is injected into the top of the first distillation column in order to reach the temperature T1.

Advantageously, the top of the first distillation column is cooled in whole or part by the liquefying agent.

Advantageously, the method comprises a step of recovering the methane in the gas state at the end of the first separation.

According to an advantageous embodiment, a second separation of the compounds of the liquid mixture recovered at the end of the first separation and composed of carbon dioxide, liquefying agent and optional hydrocarbons is carried out. This second separation is carried out by cryogenic distillation in a second distillation column after expansion of the liquid mixture in order to reach a temperature and an equilibrium pressure allowing the separation of the carbon dioxide in the form of condensate at the top of the column and the liquefying agent, supplemented with any hydrocarbons in liquid residue form at the bottom of the column.

Advantageously, the second separation is carried out by cooling the top of the second column to the condensation temperature of the carbon dioxide between −50° Celsius and −60° Celsius as a function of the pressure reached after the expansion.

Advantageously, the separation method comprises a step of recovering the carbon dioxide in the gaseous state (condensate) at the end of the second separation.

Advantageously, the method comprises a step of recovering the liquefying agent supplemented with any hydrocarbons at the end of the second separation to redirect all or some of it toward the first distillation column.

Advantageously, the liquefying agent is a linear or non-linear hydrocarbon of the alkene type of family C₃ to C₇ or a mixture of hydrocarbons from the C₃ to C₇ family.

Advantageously, the liquefying agent is a hydrocarbon or a mixture of identical hydrocarbons or with physical-chemical properties equivalent to the optional hydrocarbon or mixture of hydrocarbons present in the biogas.

The present disclosure also relates to an installation making it possible to separate all or some of a biogas in the liquid state or in the two-phase liquid/vapor state, containing methane, carbon dioxide and optionally a hydrocarbon or a mixture of hydrocarbons, implementing the separation method as described above. The installation for this purpose comprises a first distillation column intended to separate the methane from the other compounds by cryogenic distillation, the first distillation column comprising a primary feed for the injection of the liquefied biogas at an equilibrium temperature making it possible to obtain a two-phase mixture ensuring the separation of the various compounds and a secondary feed for the injection of a liquefying agent in the liquid state composed of a mixture of hydrocarbon(s) from the C₃ to C₇ family, the secondary feed being arranged to inject the liquefying agent at the top of the first distillation column, above the biogas inlet. According to an advantageous embodiment, provision may be made for the installation to further comprise a second distillation column for the separation of the compounds from the liquid mixture recovered at the end of the first separation and composed of carbon dioxide, liquefying agent and optional hydrocarbons.

The advantage of the separation method according to the present disclosure and of the associated installation is to make it possible to obtain high purity levels of the compounds separated from the biogas, that is to say on the order of 99.9% for each of the compounds (methane and carbon dioxide) and of the same order for the liquefying agent recovered at the outlet of the second separation.

The advantage of the separation method according to the present disclosure and the installation, in the case of a non-liquid biogas or one in the two-phase state, is to prevent the formation of dry ice at the top of the column during the separation of methane from the other compounds of the biogas.

### BRIEF DESCRIPTION OF THE DRAWINGS

Other features and advantages of the present disclosure will emerge from the following detailed description of embodiments of the present disclosure with reference to the appended figures, and in which:

FIG. 1 shows a schematic view of an example embodiment of an installation for separating all or some of the compounds from a biogas in the liquid state or in a two-phase liquid/vapor state implementing a separation method according to the present disclosure; and

FIG. 2 represents the main steps of a method for separating all or some of the compounds from a biogas in the liquid state or in a two-phase liquid/vapor state according to the present disclosure.

### DETAILED DESCRIPTION

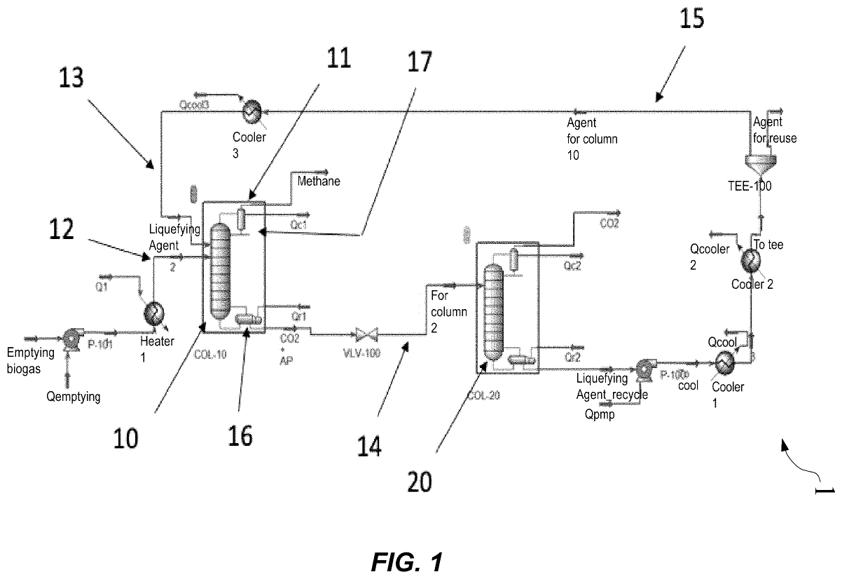

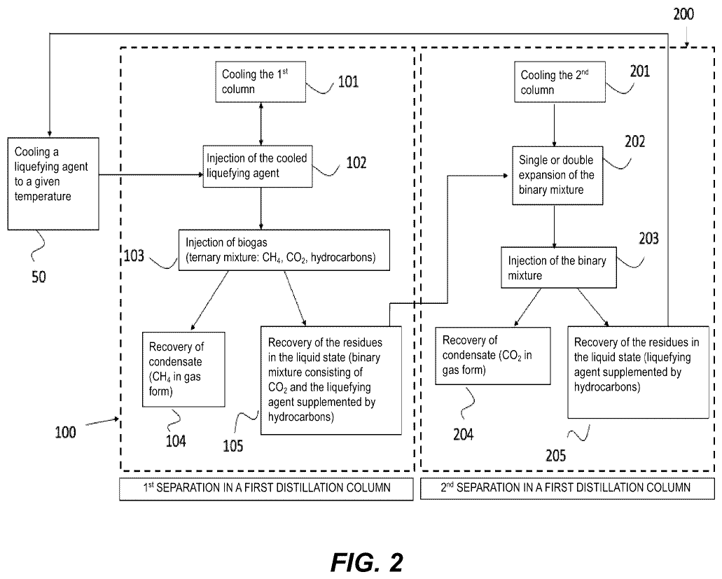

In relation to FIGS. 1 and 2, a method is described for separating the compounds from a biogas in the liquid state or in a two-phase liquid/vapor state and the installation provided for this purpose.

In the example described, the biogas to be treated contains methane, carbon dioxide as well as a hydrocarbon or a mixture of hydrocarbons from the C₃ to C₇ family, the hydrocarbon or the mixture of hydrocarbons having been introduced to ensure the prior liquefaction in all or some of the biogas. The liquefied biogas will also be referred to as the “ternary mixture” in the present disclosure, being composed predominantly of methane, carbon dioxide and a hydrocarbon. In the example described below, a liquefied biogas is considered or made predominantly liquid by n-butane (C₄). The present disclosure is of course not limited to this hydrocarbon, the following description remains transposable to a liquefied biogas or one made predominantly liquid by a hydrocarbon or a mixture of hydrocarbons from the C₃ to C₇ family other than C₄.

The separation of the compounds from the biogas is based on double distillation. The installation 1, shown in FIG. 1, thus comprises a first distillation column 10 intended to purify the biogas and a second distillation column 20 making it possible to separate the carbon dioxide from the other compounds.

The first distillation column 10 comprises theoretical plates defining the stages of the column, a packing material to promote bubbling as well as an exchange surface suitable for the vapors rising through the descending liquids, a condenser 11 fluidically connected to the first distillation column 10, at the top thereof, as well as heating means, of the reboiler type, provided at the bottom of the first column.

The first distillation column 10 further comprises a double feed: a first feed, designated the primary feed 12, for the injection of biogas, and a second feed, designated the secondary feed, 13, for the injection of a liquefying agent in the liquid state. The latter will be described in detail later. The primary feed 12 delimits the upper part of the column (top of the column) from the lower part of the column. The secondary feed 13 is arranged to allow the injection of the liquefying agent at the top of the column, advantageously at the first theoretical plate of the column, but preferably at the reflux 17 of the condenser.

The second distillation column 20 comprises an arrangement similar to that of the first column, although comprising only a single feed to inject the residues resulting from the separation operation in the first distillation column 10 and conveyed via a circuit 14 having as inlet point a tank 16 in which the residues were stored after recovery at the bottom of the first distillation column 10 and as outlet point, the second distillation column 20. It thus comprises plates defining the theoretical stages of the column, a packing material to promote the appropriate bubbling of vapors rising through the descending liquids, a condenser fluidically connected to the second distillation column 20, at the top thereof, as well as heating means, of the reboiler type, provided at the bottom of the first distillation column 10.

The installation 1 further comprises a circuit 15 for redirecting the liquefying agent supplemented by the hydrocarbon(s) initially present in the biogas to the first distillation column 10, the redirection circuit 15 comprising as inlet point the bottom of the second distillation column 20 and as outlet point, the secondary feed 13 of the first column.

According to the separation method (FIG. 2), the biogas is subjected to a first separation (step 100) to separate the methane from the other compounds (carbon dioxide and hydrocarbons). This first separation is carried out by cryogenic distillation in the first distillation column 10.

More particularly, the first separation is carried out by injecting into the first distillation column 10, as primary feed, the liquefied biogas at an equilibrium temperature making it possible to obtain a two-phase mixture allowing the separation of the various compounds (step 103).

Prior to its introduction into the first distillation column 10, the ternary mixture is heated to a temperature of between −60° C. and −50° C., the pressure being on the order of 21 bars. The mixture thus heated is introduced into the first distillation column 10 to separate the methane from the carbon dioxide and hydrocarbons. The methane constituting the lightest compound relative to the mixture of hydrocarbons and carbon dioxide rises in gas form to the top of the column while the carbon dioxide and the hydrocarbons descend in liquid form to the bottom of the column. The methane forms the condensate at the top of the first column while the mixture of hydrocarbons and carbon dioxide forms the residues at the bottom of the column.

In order to prevent the carbon dioxide from forming ice, a liquefying agent is injected via the secondary feed 13 at a temperature T1 lower than or equal to the temperature that would cause the carbon dioxide to desublimate under the operating conditions of the distillation column, in an amount proportional to the vapor flow rate of the carbon dioxide ascending to the top of the column (step 102). Advantageously, the injected liquefying agent is composed of a hydrocarbon or a mixture of hydrocarbon(s) from the C₃ to C₇ family in liquid form. Preferably, it is a linear or non-linear hydrocarbon (alkene type) or a mixture of hydrocarbons from the C₃ to C₇ family. In the example described, the liquefying agent chosen is identical to the hydrocarbon present in the biogas to be purified and which had been used to liquefy the biogas, namely n-butane (C₄). In this case, the temperature T1 at which the liquefying agent is injected will be on the order of −100° C.

Advantageously, the liquefying agent is injected at the same level as the reflux of the condenser.

Advantageously, the liquefying agent is subjected to cooling under pressure before it is injected into the top of the first distillation column 10 in order to reach the temperature T1 (step 50). In the example described, it is thus brought from ambient temperature to the temperature of −100° C. by passing through two cooling stages via exchangers, a first stage at −56° ° C. followed by a second stage at −100° C.

For the purposes of proceeding with the first separation, the top of the column is cooled beforehand to be thus brought to the condensation temperature of methane (step 101). In the example described, the top of the column is advantageously brought to the temperature of −106° ° C. for a pressure on the order of 20 bars. Advantageously, the top of the first distillation column 10 is also cooled by the liquefying agent (this cooling is shown in FIG. 2 by the double arrow between step 101 and step 102). The liquefying, cooled beforehand (step 50), is injected into the column at the temperature of −100° C. with a sufficient flow rate to generate a ternary mixture with methane, carbon dioxide and butane initially present.

Once the column has reached equilibrium, the emptying of the ternary mixture begins, using a pump. The mixture is then heated to −56° C. Once separated from the rest of the compounds, the methane is recovered (step 104) while the residue composed of carbon dioxide and of the liquefying agent supplemented by the initially present hydrocarbon is expanded after passing through the reboiler and continues the second distillation phase in the second distillation column (step 105).

In the example described, carbon dioxide is separated. To do this, the residue composed of carbon dioxide and of the mixture of hydrocarbons, in this case a mixture of butane (a mixture composed of the hydrocarbon present in the biogas and of the liquefying agent injected at the top of the column 10) is subjected to a second cryogenic distillation in the second distillation column 20 (step 200).

Likewise, prior cooling of the top of the second column is necessary before the launch of distillation (step 201). In the example described, it is set to the condensation temperature of the carbon dioxide of between −50° Celsius and −60° Celsius as a function of the pressure reached after the expansion (expansion up to 5.5 bar). Once the second column has reached its equilibrium point, the binary mixture is introduced into the second distillation column (step 203) after having first been subjected to an expansion (up to 5.5 bar) (step 202) to reach an equilibrium temperature and pressure allowing the separation of the carbon dioxide in the form of condensate at the top of the column and the liquefying agent supplemented with any mixtures of hydrocarbons in liquid residue form at the bottom of the column. During the injection of the binary mixture into the second distillation column 20, the carbon dioxide undergoes vaporization and rises to the top of the second column in the gas state while the mixture of hydrocarbons descends in the liquid state to the bottom of the second distillation column 20.

Once separated from the rest of the compounds, the carbon dioxide is recovered (step 204) while the mixture of hydrocarbons is recovered at the bottom of the distillation column in order to be optionally rerouted at least in part to the first distillation column 10 or a buffer storage tank in order to optionally be reused to liquefy a biogas (step 205).

In the embodiment described above, the separation of all the compounds constituting the biogas is carried out. However, it may be provided that only some of the compounds are separated, in order to recover only part of the biogas, in this case methane.

Likewise, in the embodiment described above, the liquefied biogas subjected to the separation method according to the present disclosure is a ternary mixture composed predominantly of methane, carbon dioxide and a hydrocarbon of the C₃ to C₇ family. However, it may be provided that the biogas composed predominantly of methane and carbon dioxide, the separation method, and the installation can be implemented to purify such a biogas without departing from the scope of the present disclosure. Likewise, the biogas fed into the column is a mixture in the liquid state or in the two-phase liquid/vapor state.

The present disclosure is described in the foregoing by way of example. It is understood that a person skilled in the art is able to produce different variant embodiments of the present disclosure without departing from the scope of the invention as defined by the claims.

1. A method for separating all or some of the compounds from a biogas in a liquid state or in a two-phase liquid/vapor state, containing methane, carbon dioxide wherein a first separation for separating the methane from the biogas is carried out by cryogenic distillation in a first distillation column comprising a column top brought to the condensation temperature of the methane at a given pressure, the first separation being carried out by injecting into the first distillation column:
in a primary feed, the biogas liquefied at an equilibrium temperature making it possible to obtain a two-phase mixture ensuring the separation of the various compounds,
in a secondary feed, a liquefying agent in the liquid state composed of a hydrocarbon or a mixture of hydrocarbons from the C₃ to C₇ family, the liquefying agent being injected at the top of the first distillation column, above a biogas inlet, at a temperature T1 lower than or equal to the carbon dioxide desublimation temperature at a given pressure of the first distillation column and in an amount proportional to the vapor flow rate of the carbon dioxide ascending to the top of the first distillation column,
and wherein a second separation of the compounds of the liquid mixture recovered at the end of the first separation and comprising carbon dioxide and the liquefying agent is carried out by cryogenic distillation in a second distillation column after expansion of the liquid mixture to reach a temperature and an equilibrium pressure allowing separation of the carbon dioxide in the form of condensate at the top of the second distillation column and the liquefying agent in liquid residue at the bottom of the second distillation column.

2. The method of claim 1, wherein the liquefying agent is injected into the first distillation column at a level at which methane reflux is removed from the first distillation column.

3. The method of claim 2, wherein the liquefying agent is injected into the first distillation column at a temperature T1 on the order of −100° C.

4. The method of claim 3, wherein the liquefying agent is subjected to cooling in two stages under pressure before it is injected into the top of the first distillation column to reach the temperature T1.

5. The method of claim 4, wherein the top of the first distillation column is cooled at least in part by the liquefying agent.

6. The method of claim 5, further comprising recovering the methane in a gas state at the end of the first separation.

7. The method of claim 6, wherein the second separation is carried out by cooling the top of the second distillation column to the condensation temperature of the carbon dioxide between −50° Celsius and −60° Celsius as a function of the pressure reached after the expansion.

8. The method of claim 7, further comprising recovering the carbon dioxide in a gas state at the end of the second separation.

9. The method of claim 1, further comprising recovering the liquefying agent at the end of the second separation and redirecting at least some of the recovered liquefying agent to the first distillation column.

10. The method of claim 1, wherein the liquefying agent is a linear or non-linear alkene hydrocarbon of the C₃ to C₇ family or a mixture of hydrocarbons from the C₃ to C₇ family.

11. The method of claim 13, wherein the liquefying agent comprises the same hydrocarbon or mixture of hydrocarbons present in the biogas.

12. An installation enabling the separation of at least some of a biogas in a liquid state or in a two-phase liquid/vapor state, containing methane and carbon dioxide, the installation comprising:
a first distillation column intended configured to separate the methane from the other carbon dioxide by cryogenic distillation, the first distillation column comprising a primary feed for the injection of the liquefied biogas at an equilibrium temperature making it possible to obtain a two-phase mixture ensuring the separation of the methane from the carbon dioxide and a secondary feed for the injection of a liquefying agent in the liquid state composed of a hydrocarbon or a mixture of hydrocarbons from the C₃ to C₇ family, the secondary feed being arranged to inject the liquefying agent at the top of the first distillation column, above a biogas inlet; and
a second distillation column for the separation of the carbon dioxide from a liquid mixture recovered at the end of the first separation and comprising the carbon dioxide and the liquefying agent.

13. The method of claim 1, wherein the biogas further comprises a hydrocarbon or a mixture of hydrocarbons from the C₃ to C₇ family, and wherein the liquid mixture recovered at the end of the first separation further comprises the hydrocarbon or the mixture of hydrocarbons from the C₃ to C₇ family, and the liquid residue at the bottom of the second distillation column further comprises the hydrocarbon or the mixture of hydrocarbons from the C₃ to C₇ family.

14. The method of claim 13, wherein the liquefying agent exhibits the same physical-chemical properties exhibited by the hydrocarbon or mixture of hydrocarbons present in the biogas.

15. The method of claim 1, wherein the liquefying agent is injected into the first distillation column at a temperature T1 on the order of −100° C.

16. The method of claim 1, wherein the liquefying agent is subjected to cooling in two stages under pressure before it is injected into the top of the first distillation column to reach the temperature T1.

17. The method of claim 1, wherein the top of the first distillation column is cooled at least in part by the liquefying agent.

18. The method of claim 1, further comprising recovering the methane in a gas state at the end of the first separation.

19. The method of claim 1, wherein the second separation is carried out by cooling the top of the second distillation column to the condensation temperature of the carbon dioxide between −50° Celsius and −60° Celsius as a function of the pressure reached after the expansion.

20. The method of claim 1, further comprising recovering the carbon dioxide in a gas state at the end of the second separation.
